(19) United States
(12) Patent Application Publication
(10) Pub. No.: US 2011/0256191 A1
(43) Pub. Date: Oct. 20, 2011

(54) COMPOSITION

(76) Inventor: Aneliya Nikolova Zdravkova, Wirral (GB)

(21) Appl. No.: 13/127,812

(22) PCT Filed: Nov. 10, 2009
(86) PCT No.: PCT/EP09/64929

§ 371 Date: Jun. 27, 2011

(30) Foreign Application Priority Data
Nov. 14, 2008 (EP) 08169099.2

(51) Int. Cl. A61K 8/73; A61Q 5/12; A61K 8/06

(52) U.S. Cl. 424/401; 424/70.12

(57) ABSTRACT

Leave-on hair care composition comprising an oil in water emulsion, the emulsion comprising a silicone gum of molecular weight of from 800 000 to 5 000 000 cSt, a nonionic or anionic polysaccharide and a low molecular weight solvent for the silicone gum.

The present invention relates to a composition for achieving hair shine.

Despite the prior art there remains the need for attractive leave-on hair care compositions for creating shiny hair.

Accordingly, and in a first aspect, the present invention provides a hair care composition according to claim 1.

Using such a polysaccharide one can achieve compositions with greater hair alignment properties without high levels of silicone. The compositions also achieve good shine and tactile attributes.

The viscosity of the silicone is measured on a Brookfield RV4 Viscometer at 25° C. using spindle no. 4 at 100 rpm.

Preferably, the low molecular weight solvent is selected from silicones such as low molecular weight silicones, such as dimethicone having a viscosity of 10 cSt or less; cyclic silicones having from 3 to 6 carbons, e.g. pentasiloxane; hydrocarbons having 16 or fewer carbons, e.g. isododecane, isoparaffin, (C13-16); or phenyltrimethicone.

Preferably, the polysaccharide is xanthan, carrageenan, hydroxyethyl cellulose and derivatives thereof.

Preferably, the polysaccharide is present at from 0.1 to 5% wt. more preferably from 0.15 to 0.8% wt. of the composition.

Preferably, the emulsion comprises droplets of average size from 1 to 50, more preferably from 2 to 25 and most preferably from 3 to 24 microns.

Preferably, the composition according to the invention comprises less than 4% wt. silicone, more preferably less than 2% wt. silicone.

The composition according to the invention is preferably a serum composition and comprises less than 5% wt. anionic surfactant and more preferably less than 2% wt. anionic surfactant.

The composition according to the invention comprises water. Preferably, the composition comprises from 10 to 90% wt. water, more preferably from 25 to 75% wt. water.

A leave-on composition is one which is intended to be left on the hair after application as opposed to one which is washed or rinsed off after application.

In a second aspect the present invention provides a method for achieving hair alignment by applying to the hair a composition according to the first aspect.

Embodiments of the invention will now be described with reference to the following non-limiting examples.

### EXAMPLE 1

The following are formulations according to embodiments of the invention. They are made by standard processes.

| | 1 | 2 | 3 | 4 |
|---|---|---|---|---|
| Water | 34.29 | 34.29 | 44.24 | 44.24 |
| Glycerol | 29.9 | 29.9 | 38.45 | 38.45 |
| PEG 400 | 3.5 | 3.5 | 4.5 | 4.5 |
| NaCl | 1.4 | 1.4 | 1.8 | 1.8 |
| C₇H₅NaO₂ | 0.7 | 0.7 | 0.9 | 0.9 |
| Natrosol 250 HHR | 0.21 | | 0.11 | |
| Carageenan | | 0.21 | | 0.11 |
| DC1413 | 24 | 24 | 8 | 8 |
| Sepigel 305 | 6 | 6 | 2 | 2 |

Ingredients in Bold represent the oil phase.

### EXAMPLE 2

Alignment e-panel data. Pictures of the switches, treated with the formulations and the control serums, were taken and were used for alignment measurement. The pictures were evaluated for alignment by panels of volunteers from our laboratory following an established alignment protocol.

| Product | Alignment | SD |
|---|---|---|
| Serum A | 1.641 | 0.9738 |
| Serum B | 6.7692 | 0.8155 |
| 1 | 7.8077 | 0.5689 |
| 2 | 7.5513 | 1.0507 |
| 3 | 6.8846 | 0.35 |
| 4 | 7.8205 | 0.677 |

Serum A is a Sunsilk serum with 9.3% wt. silicone.
Serum B is Charles Worthington ‘Really Shiny Gloss Polish’ serum with 6.9% wt. silicone

1. Leave-on hair care composition comprising an oil in water emulsion, the emulsion comprising a silicone gum of molecular weight of from 800 000 to 5 000 000 cSt, a nonionic or anionic polysaccharide and a low molecular weight solvent for the silicone gum.

2. Composition according to claim 1 wherein the low molecular weight solvent is selected from silicones such as low molecular weight silicones, cyclic silicones having from 3 to 6 carbons, hydrocarbons having 16 or fewer carbons and phenyltrimethicone.

3. Composition according to claim 1 wherein the polysaccharide is xanthan, carrageenan, hydroxyethyl cellulose and derivatives thereof.

4. Composition according to claim 1 wherein the emulsion comprises droplets of average size from 1 to 50, more preferably from 2 to 25 and most preferably from 3 to 24 microns.

5. Composition according to claim 1 which comprises less than 4% wt. silicone.

6. Composition according to claim 1 which comprises less than 5% wt. anionic surfactant.
